(19) United States
(12) Patent Application Publication
(10) Pub. No.: US 2012/0185016 A1
(43) Pub. Date: Jul. 19, 2012

(54) PROCESS AND APPARATUS FOR TREATING BIOLOGICAL ORGANISMS

(75) Inventors: Michael L. Weiner, Estero, FL (US); Robert W. Gray, Rochester, NY (US); Stuart G. MacDonald, Pultneyville, NY (US); Jack A. Tuszynski, Edmonton (CA); Amara D. Angelica, Boulder Creek, CA (US); Kendrick Goss, Lexington, MA (US)

(73) Assignee: Biomed Solutions LLC, Pittsford, NY (US)

(21) Appl. No.: 13/331,689

(22) Filed: Dec. 20, 2011

Related U.S. Application Data
(63) Continuation-in-part of application No. 12/155,824, filed on Jun. 10, 2008, now abandoned.
Application No. 12/155,824 is a continuation of application No. 11/066,418, filed on Feb. 25, 2005, now abandoned.
Application No. 11/066,418 is a continuation-in-part of application No. 09/930,364, filed on Aug. 15, 2001, now abandoned.

(51) Int. Cl. A61N 1/36

(52) U.S. Cl. 607/62

(57) ABSTRACT

Disclosed in this specification is an apparatus and method for treating a biological organism comprising a device for emitting and delivering energy to the biological organism, a programmable controller for varying the type and amount of energy emitted, and apparatus for sensing a condition of the biological organism.

### CROSS-REFERENCE TO RELATED APPLICATIONS

This application is a continuation-in-part of co-pending U.S. patent application Ser. No. 12/155,824, filed Jun. 10, 2008, which is a continuation of Ser. No. 11/066,418, filed Feb. 25, 2005, now abandoned, which is a continuation-in-part of U.S. patent application Ser. No. 09/930,364, filed on Aug. 15, 2001, now abandoned, which applications are incorporated herein by reference in their entirety.

### FIELD OF THE INVENTION

This invention relates, in one embodiment, to a treatment of biological organisms with various forms of energy, particularly electromagnetic energy.

### BACKGROUND

It is known that the application of certain electromagnetic energies and signals can change the biological effectiveness of fluids including water. See Dr. Alan Halls' book entitled “Water, Electricity and Health”, Hawthorn Press, 1997 and references cited therein, as well as the papers “Digital Recording/Transmission of the Cholinergic Signal” by Dr. J. Benveniste, et. al. and references therein.

Similarly, the application of exterior photonic and other electromagnetic energy to a body for therapeutic purposes is also known. See, for example, WO9417406 and U.S. Pat. No. 6,541,978 to Benveniste, the contents of which are hereby incorporated by reference. Benveniste determined the characteristic electric signal of an isolated substance (e.g. caffeine, distilled water, ionophoretic-calcium, ovalbumin, propranolol) and then impose that signal on a biological system (e.g. a perfused guinea-pig heart, bacterium coli, streptococci, staphilocci). Surprisingly, Beneveniste discovered at least certain physiological effects of the isolated substance (e.g. caffeine) can be imposed on the biological system (e.g. perfused guinea-pig heart) by application of the substance's characteristic electric signal. This physiological effect was imposed without treatment with the substance itself.

### SUMMARY OF THE INVENTION

In accordance with this invention, there is provided a process for treating a biological organism comprising the steps of measuring a first electromagnetic energy signal characteristic of a biological sample while experiencing the effect of a therapeutic agent; measuring a second electromagnetic energy signal characteristic of the biological sample while not experiencing the effect of the therapeutic agent; comparing the first and second electromagnetic energy signals and determining a third electromagnetic energy signal that, when superimposed on the second electromagnetic energy signal, results in a fourth electromagnetic energy signal that is substantially identical to the first electromagnetic energy signal. The third electromagnetic energy signal is then delivered to a biological organism.

### BRIEF DESCRIPTION OF THE DRAWINGS

The present invention is disclosed with reference to the accompanying drawings, wherein:

FIG. 1 is a schematic of one implantable device of this invention disposed within a patient;

FIG. 2 is a block diagram of one possible process for determination and subsequent utilization of an energy pattern;

FIGS. 3 through 8 are schematic diagrams of various arrangements of one or more implantable devices disposed within a patient;

FIG. 9 is a flow diagram of the operation of the device of FIG. 4;

FIGS. 10, 11, and 12 are graphs of some of the energy patterns delivered to a patient in one of the processes of this invention;

FIG. 13 shows a shunt configuration;

FIG. 14 is a schematic showing utilization of the invention in a tube or pipe;

FIG. 15 is a schematic showing utilization of the invention in a fluid holding vessel;

FIG. 16 is a schematic diagram of three types of energy emitting devices in accordance with embodiments of the invention;

FIG. 17 is a schematic diagram of another energy emitting device in accordance with embodiments of the invention;

FIG. 18 is a block diagram of process for treatment of diseased cells;

FIG. 19 is a schematic diagram of a stent with a light emitting coating in accordance with embodiments of the invention;

FIG. 20 is a schematic diagram of two devices for treatment of congestive heart failure; and

FIG. 21 is a schematic illustration of a device for interrogating cellular components.

Corresponding reference characters indicate corresponding parts throughout the several views. The examples set out herein illustrate several embodiments of the invention but should not be construed as limiting the scope of the invention in any manner.

### DETAILED DESCRIPTION

One embodiment of this invention involves the application of electromagnetic energy to an organism. It is known that very minute alterations to molecules and fluids, such as blood or water, can have dramatic therapeutic effects, and that it is possible to digitize the method for effecting the alterations of these treatments and transmit them electronically so that they can be repeated with high precision at a later time and if necessary in a different place. See, for example, U.S. Pat. No. 6,541,978 to Benveniste. As a result, complex diagnostics, including imaging and chemical analyses, can be conducted of tissue or fluid samples at a remote site, and a patient prescription provided for treating the situation that can be transmitted to the patient location and administered locally.

It is noteworthy that molecules can activate their corresponding receptor sites without physical contact. See, e.g., an article by C. W. Smith, “Electromagnetic effects in humans,” in Biological Coherence and Response to External Stimuli, Frohlich H (editor), Springer-Verlag, Berlin, pages 205-232. Reference also may be had to James L. Oschman's book Energy Medicine: The Scientific Basis (Churchill Livingston, New York, N.Y., 2000) and a book published in 1957 by A. Szent-Gyorgyi entitled Bioenergetics, published by Academic Press, New York. In one process of this invention, the energy patterns from signal molecules are used without their corresponding drugs to treat the receptor sites.

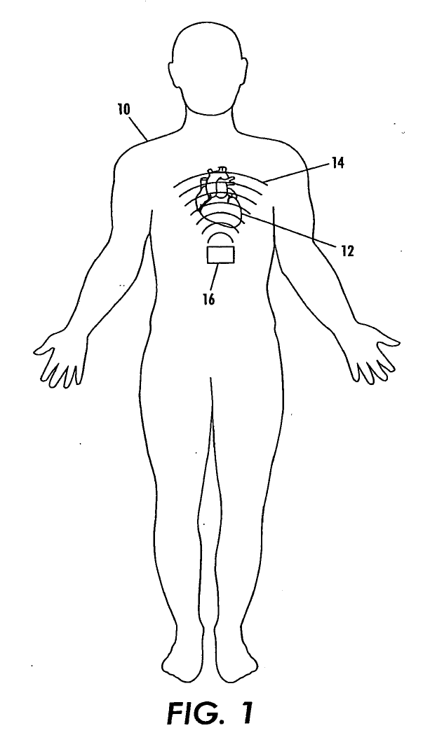

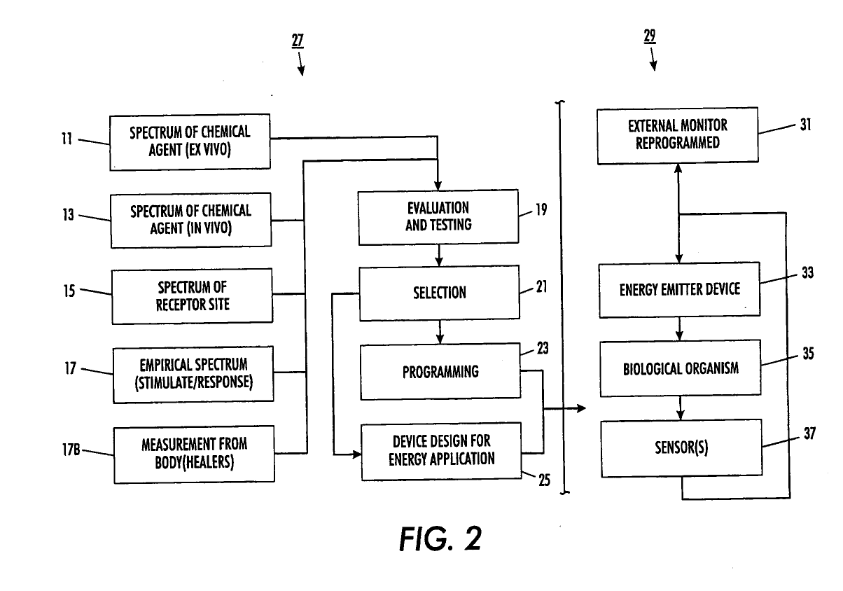

Referring to FIG. 1, in one embodiment, the emitter 16 is utilized to effect a process for treating the body 10. In this process, one first determines the electromagnetic pattern of a biological process within body 10. This energy pattern determination may be made, e.g., by the process depicted in FIG. 2. Once the electromagnetic or other energy pattern has been determined, a portion of said energy pattern may be directly applied within the body 10. In one embodiment, the energy emitted by emitter 16 varies with time in either its frequencies and/or amplitudes and/or phases. In another embodiment, the energy spectrum emitted by emitter 16 varies with time. Thus, by way of illustration, one may transmit the spectra of a drug as it dissolves in the organism and interacts with the organism over time.

In one embodiment, the energy emitted by emitter is a combination of energies selected from the group consisting of photonic energy, vibratory energy, electrical energy, and mixtures thereof, provided that, in this embodiment, at least two of such energies are emitted.

In one embodiment, a first energy signal of a biological sample is measured. Beneveniste (U.S. Pat. No. 6,541,978) describes a process for determining “from a biological and/or chemical active element such as a chemical compound, a cell or a micro-organism, or from a substance containing this active element such as a purified preparation, a biological sample, or a living being, an ‘electromagnetic signal characteristic of the biological and/or chemical activity or of the biological and/or chemical behavior’ of said substance.” In one embodiment, the chemical compound or biological organism is placed in a chamber and subjected to certain conditions that permits the electromagnetic signal that is characteristic of the organism or compound to determine according to the techniques of Beneveniste. By way of illustration, Beneveniste determines the electromagnetic signal of caffeine and then imposes the same electromagnetic signal on a guinea-pig heart. A control experiment was conducted which (a) exposed a guinea-pig heart directly to caffeine (a Langendorff experiment) and (b) monitored a drug/signal-free control heart. The heart that was subjected to the electromagnetic signal of caffeine behaved in a fashion similar to that of the heart that was directly exposed to caffeine and in a fashion unlike the control heart. Beneveniste scanned across a variety of wavelengths to determine the characteristic electromagnetic signal.

By way of further illustration, one may use energy of from about 1 to about 3 hertz to regenerate nerves. One may use an energy of from about 5 to about 9 hertz to promote bone growth. One may use an energy of about 10 hertz to heal ligaments. Generally, energies of 15, 20, and 72 hertz decrease skin necrosis, stimulate capillary formation, and cause the proliferation of fibroblasts. Energies of 25 and 50 hertz promote synergistic effects with nerve growth factor. In general, the use of energies from about 1 to about 100 hertz promotes healing of many bodily parts.

In one aspect of this embodiment, millimeter and/or centimeter wavelength energy is used. In general, this energy has a frequency of from about 30 to about 300 gigahertz. In some papers, reference to “millimeter waves” refers to frequencies around 60 gigahertz.

In one embodiment, the energy utilized in the process of this invention has a frequency of at least 1,000 gigahertz (one terahertz) and is believed to cause deoxyribonucleic acid to resonate. In this embodiment, a multiplicity of different frequencies, each of which has a frequency of at least one terahertz, are used.

FIG. 2 is a flow diagram which illustrates one embodiment of the energy pattern determination process of this invention. In step 11 of the process illustrated in FIG. 2, the spectrum (characteristic electromagnetic signal) of a therapeutic agent is determined by spectral analysis, or by reference to standard tables of the spectrum of the agent. In one embodiment, an electromagnetic signal characteristic of the biological and/or chemical activity of a therapeutic agent is determined using the chamber described in U.S. Pat. No. 6,541,978.

Referring again to FIG. 2, and in the embodiment depicted therein, in step 11 the spectrum of a chemical agent is determined ex vivo, outside of a biological organism. Alternatively, or additionally, one may determine the spectrum of a chemical agent in vivo in step 13 by conventional means. In both step 11 and 13, one may determine the spectrum of only one agent, or of two or more agents, in various combinations and at various concentrations. Alternatively, or additionally, one may determine the spectrum of one or more agents over a period of time. As is known to those skilled in the art, a drug within a biological organism will change its physical and/or chemical identity, due to dissolution in one or more solvents and/or reaction with one or more agents within the body. As the physical and chemical properties of the drug change, so does its spectrum.

Alternatively, and as is illustrated in step 15, one may determine the spectrum response of a receptor site to various stimuli, including stimulation by drugs as well as stimulation by application of various energy patterns or by combinations. Alternatively, one may determine the spectrum of the biological organism, over time, as it is exposed to a drug. By trial and error, one may determine what combination of stimuli produce the desired receptor response.

Alternatively, as is illustrated in FIG. 2, step 17, one may determine a therapeutic energy pattern by subjecting the body and/or individual organs, tissues, bodily fluids, cells, cells in culture to various energy patterns and recording the response. Alternatively, as illustrated in FIG. 2, step 17B, one may determine a therapeutic energy pattern by measuring the energy patterns of a healthy body, and/or individual organs, tissues, bodily fluids, cells.

In one embodiment, a second energy signal of a biological sample is measured once one has determined a desired energy signal (first energy signal) produced by a specified drug or combination of drugs. One may then evaluate which combination of energy pattern stimuli will produce the same response in step 19 of the process.

A electromagnetic signal characteristic of the biological and/or chemical activity or of the biological and/or chemical behavior' of the biological organism is determined while it is not under the effects of the therapeutic agent. Techniques for determining such a characteristic electromagnetic signal are known to those skilled in the art. See U.S. Pat. No. 6,541,978.

Referring again to FIG. 2, once a desired (first energy signal) energy spectrum, or portion thereof, or combination of one or more such spectra, is identified, the second energy signal may be evaluated in step 19 against the candidate spectra (the first energy signal). The response of a biological body, or a portion thereof, can be determined, and a correlation can be made between the use of a specified (third) spectrum and/or spectra and the response of the organism. The third spectrum is determined such that the mathematical sum of the third and first energy signals will produce a fourth energy spectrum that is substantially identical to the first energy signal, thereby providing a net energy signal that approximates the effect of the therapeutic agent that produced the first energy signal. The third spectrum is designed to add to the intensity of the wavelengths of the second energy signal at those wavelengths where addition is necessary to mimic the first energy signal. The third spectrum is further designed to subtract from the intensity of those wavelengths of the second energy signal where subtraction is necessary using phase cancellation techniques. Phase cancellation is achieved by transmitting an inverse (180 degrees out of phase) signal at the same frequency as a detected signal.

Thereafter, in step 21 of the process, a (fourth) spectrum and/or spectra may be determined for any particular condition to be treated in the biological organism and information about this determined spectrum/spectra may be incorporated into a program in step 23. In step 25, the program may be incorporated into a device which is capable of sensing the condition within the biological organism, selecting the appropriate spectrum/spectra from its database, emitting such energy pattern and directing it to the appropriate site within the organism, sensing the response of the living organism to such emission, modifying such emission as appropriate, and/or ceasing such emission as appropriate.

In portion 27 of the process, which is comprised of steps 11 through 25, the steps necessary to identify the appropriate energy pattern are described. In portion 29 of the process, comprising steps 31 through 37, the steps necessary to apply the selected energy pattern to the living organism are described.

In step 31 of the process, which is optional, one may utilize an external monitor/reprogrammer for bi-directional communication between the implanted device and the outside world. With such a monitor/reprogrammer, one can visually observe indicia of the state of biological organism and, as appropriate, change the program of the implanted device.

The external monitor/reprogrammer is operatively connected to the implanted energy device of step 33 which, in response to external stimuli and/or in vivo stimuli provided by the biological organism, provides energy to biological organism of step 35. In one embodiment, depicted in step 37, a sensor which can monitor the response of the living organism to the applied energy and, with use of a programmable computer (not shown), continually modifies the energy delivered to the organism. The connection between the external monitor/reprogrammer 31 and the energy device may be direct, or it may be indirect. In one embodiment, the connection is indirect and is made, e.g., by means of transceivers.

### Exemplary Embodiments

Resistant myofascial pain can be treated with microcurrent of specific frequencies, as is disclosed in a 1998 article by C. McMakin entitled “Microcurrent treatment of myofascial pain in the head, neck, and face” published in Topics in Clinical Chiropractic 5(1):29-35.

In one embodiment, the first characteristic electromagnetic signal of a biological sample experiencing myofascial pain is determined. Likewise, the second characteristic electromagnetic signal of tissue not experiencing myofascial pain is determined. For example, a therapeutic agent may be present which results in a low pain state. By way of further illustration, the organism may be emitting an unknown therapeutic agent that causes a decrease in myofascial pain. The first and second signals are compared and a third signal is determined which, when mathematically added to the first signal, will produce a fourth signal that closely approximates the second signal. This third signal is then applied to an organism that corresponds to the biological sample. For example, if the biological sample was human tissue, then a corresponding biological organism is a human being. This human being may be the same or different than the human being from which the biological sample originated.

FIG. 1 is a schematic of one implantable device of this invention. Referring to FIG. 1, and in the embodiment depicted therein, it will be seen that an energy emitter 16 is implanted into a biological organism 10, preferably in the proximity of an organ 12. In the embodiment depicted, the emitter 16 emits photonic or other electromagnetic energy 14 onto organ 12. The energy 14 may, e.g., be electrostatic, magnetostatic, acoustic, or very low frequency (VLF) through ultraviolet electromagnetic signals.

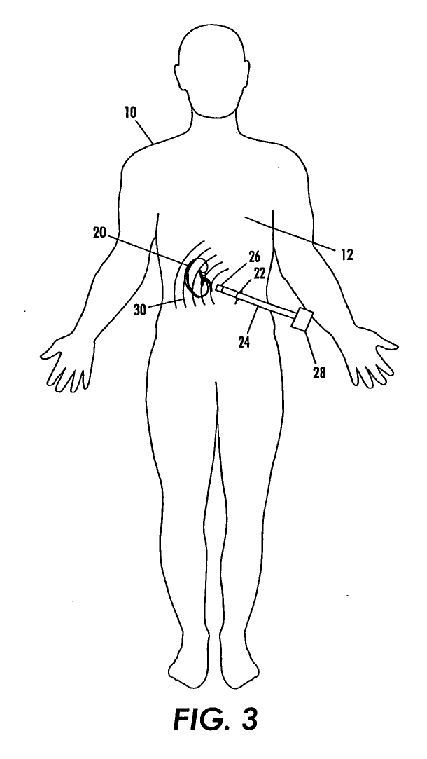

In another embodiment of this invention, illustrated in FIG. 3, an emitter 26 is attached to the end of a catheter 24 and is controlled by a controller 28. In the embodiment depicted in this FIG. 3, the catheter is inserted into body 10 through an incision 22. The organ 20 is then irradiated with the electromagnetic energy 30. An operator, not shown, may control the electromagnetic energy by adjusting parameters of the controller 28.

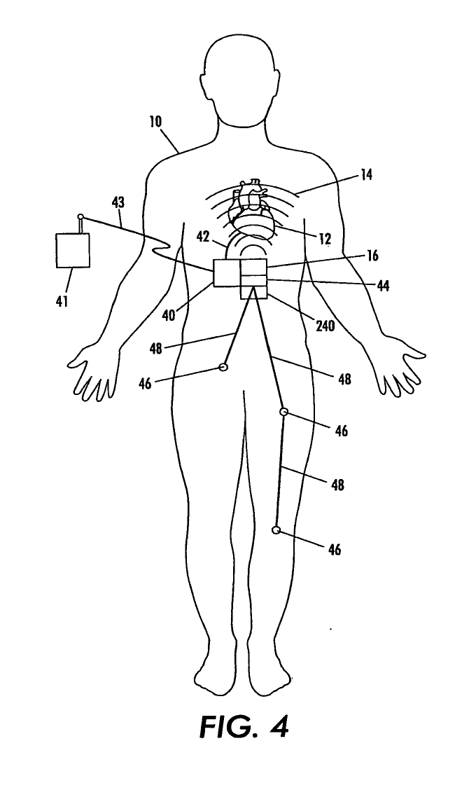

In another embodiment of this invention, illustrated in FIG. 4, an emitter 16 is an augmentation module connected to an implanted heart pacemaker 40; in the embodiment, the pacemaker 40 is connected via lead 42 to the heart 12. This augmentation module may be attached to the pacemaker 40 at any future date after the pacemaker 40 has been implanted without removal or otherwise replacement of the original pacemaker 40. Alternatively, the augmentation module may be implanted at the same time as the pacemaker 40. In either situation, the augmentation unit may be detached from the pacemaker 40 and removed from the body 10 at any time without significant disruption of the pacemaker 40. A controller with a programmable logic unit 44 is connecter to the emitter 16 and the pacemaker 40. The controller 44 also has communication means 48 to implanted sensors 46. The emitter 16 may be activated by the analyses of the sensors' input and comparison to threshold conditions or comparison to a programmable database of deleterious conditions. The emitted energy 14 may be adjusted from very low frequency to ultraviolet or terahertz range frequency programmatically through the controller's programmable logic unit 44. The emitted electromagnetic or vibrational energy signals produced by the augmentation modules may be a reproduction of the natural energy signals emitter from a healthy organ. In this way, a healthy signal may reinforce a non-healthy organ as well as to propagate a healthy signal to other organs.

Referring to FIG. 4, the sensors 46 are capable of determining the electromagnetic pattern and/or other physiological and/or biochemical and/or biophysical parameter of any portion of the body 10 while such body is functioning. One may determine the electromagnetic pattern of such body when, e.g., the liver is functioning properly. One may determine the electromagnetic pattern of such body when, e.g., the liver is not functioning properly. One may, e.g., determine the electromagnetic pattern of the heart in relation to diagnostic indicators of susceptibility to arrhythmias of various kinds. One may, e.g., determine the electromagnetic pattern of such body when the liver is exposed to one or more drugs, or to heat, or to any treatment. By making these measurements, one can correlate the optimum performance of, e.g., the liver with optimum electromagnetic patterns. Similar correlations can be made with other organs and/or bodily processes. In one embodiment, a correlation is made to an anti-inflammatory drug such as nabumetone, ibuprofen, and the like.

Once such correlations have been made, using the methods disclosed herein or by reference to research studies conducted by others, one can deliver to the patient, via emitter 16, that portion of the spectral pattern which is advantageous to the patient at times when it is advantageous to the patient. Thus, e.g., the sensors 46 can determine when, e.g., the liver is malfunctioning and deliver the required electromagnetic radiation to the patient, either alone and/or in combination with one or more drugs, until the liver is functioning properly.

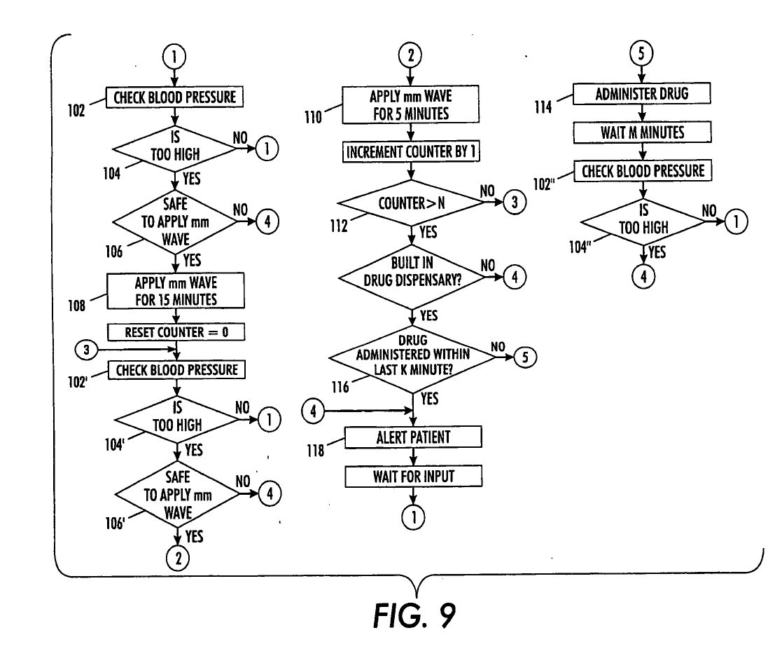

In the embodiment depicted in FIG. 4, an implantable drug dispenser 240 is operatively connected to the controller 44 and, as required, delivers one or more drugs in response to the commands of such controller 44. As will be apparent to those skilled in the art, the process depicted in FIG. 9 may be effected by the device depicted in FIG. 4.

In one embodiment, as depicted in FIG. 4, the sensors 46 are so constructed and situated as to detect energy patterns in the environment, external to the body 10. Controller 44 can analyze such patterns and can determine if such external energy patterns are disruptive to the body 10 or to the treatment currently administered. If such a disruptive external energy pattern is detected, the controller 44 may change the energy pattern emitted from emitter 16 or halt the administration of treatment until the disruptive external energy patterns are no longer detected and/or notify the patient through communications device 41 using communication channel 43. Communications channel 43 may be, e.g. by radio frequency means.

How the energy pattern of any particular drug, or combinations of drugs, or how combinations of drugs and electromagnetic fields, changes over time may be stored within the controller 44 of FIG. 4. The response of the patient's body to various portions of such energy patterns may be determined by the sensors 46 and the controller 44. In many cases, it will be determined that a certain portion of the spectral pattern, and/or its combination with one or more drugs, advantageously affects the patient's body. In other cases, it may be determined that a certain portion of the spectral pattern, and/or its combination with one or more drugs, disadvantageously affects the patient's body. The device of FIG. 4 will be capable of determining, at any particular point in time, which portion, if any, of the energy pattern and/or drug should be applied at that point in time. Thereafter, by monitoring the patient's reaction to the administered energy pattern(s) and/or drug(s), the controller 44 can cause the emitter 16 or the implantable drug dispenser 240 connected to the controller 44 to modify the energy pattern(s).

If, for example, a drug is being administered which, at a particular point in time, is producing a disadvantageous energy pattern, the emitter 16 may emit one or more interfering and/or phase shifted and/or phase inverted and/or complementary energy patterns which, after they interact with the energy pattern produced by such drug, or with the response of the receptor molecules the drug is acting upon, produce the desired energy pattern and/or lack thereof.

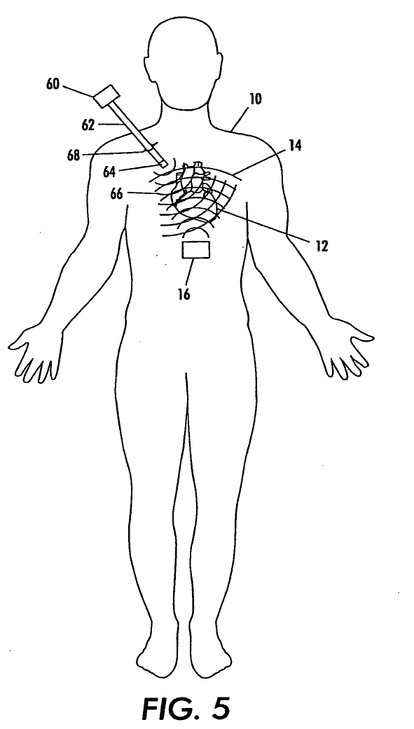

In another embodiment of this invention, illustrated in FIG. 5, an emitter 16 implanted in body 10 emits electromagnetic energy 14 onto or within an organ 12. Additionally, a probe 62 with an emitter 64 at the insertion tip of a catheter is inserted into the body 10 through incision 68. The inserted probe emitter 64 emits electromagnetic energy 66 onto the organ 12 from a different orientation than that of emitter 16. The electromagnetic energy 66 which is emitted from the probe emitter is controlled via controller 60 and need not have the same characteristics as the electromagnetic energy emitted from emitter 16.

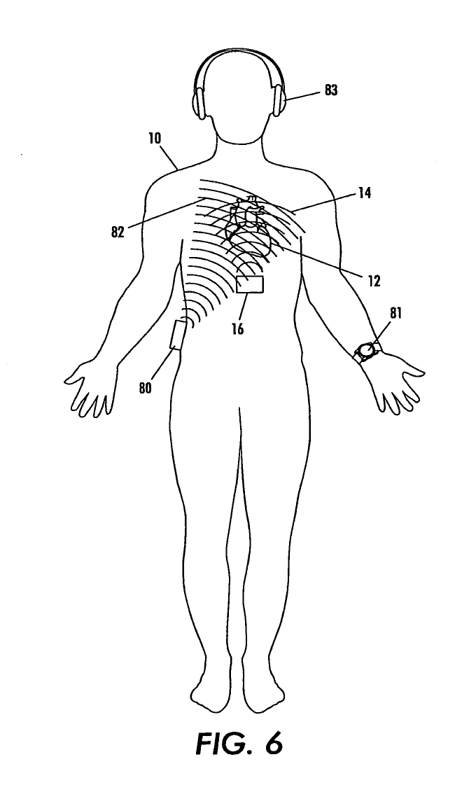

FIG. 6 illustrates an embodiment in which an emitter 16 implanted in body 10 emits electromagnetic energy 14 onto an organ 12, and an external device 80 delivers vibratory energy 82 to the organ 12. In one aspect of this embodiment, emitter 16 continually emits energy, whereas external device 80 intermittently emits energy. Other external devices 81 and 83 may also deliver various energy patterns to the body 10.

In one embodiment, illustrated in FIG. 6, electromagnetic energy may be delivered through a device located outside of the patient's body, such as a watch 81 and/or external appliances 80 and/or 83 and/or in glasses frames (not shown) ankle bracelets (not shown), etc.

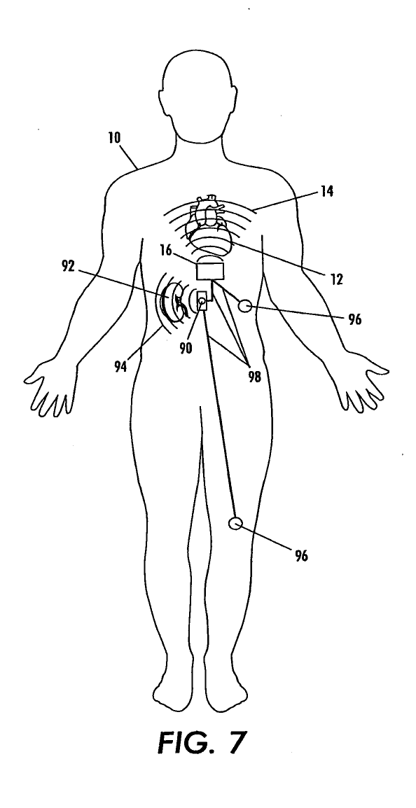

In another embodiment of this invention, illustrated in FIG. 7, an electromagnetic emitter 16 implanted in body 10 emits electromagnetic energy 14 onto an organ 12. Additionally, a vibrational energy emitter 90 is also implanted into body 10 and delivers vibrational energy 94 to organ 92. The emitter 90 also consists of a sensor element. Additionally, other sensor devices 96 are implanted into body 10. All of such implanted devices are in communication through communication channels 98 to form a network. The communication means may be through fiber optic cables, wires, shielded wires, wireless or other means. The implanted devices 16 and 90 are so constructed as to contain programmable logic units suitable for analyzing signals from other implanted devices and from sensors 96 and to initiate the adjustment of adjustable parameters of any implanted device in the network. The emitters are so designed as to allow for multiple frequencies and intensities to be emitted at the same time including a carrier and pulsed waves combined. Each of these implanted devices is so constructed as to allow the addition of other implanted or external devices or the removal of said devices from the network of devices without the interruption of other devices in the network.

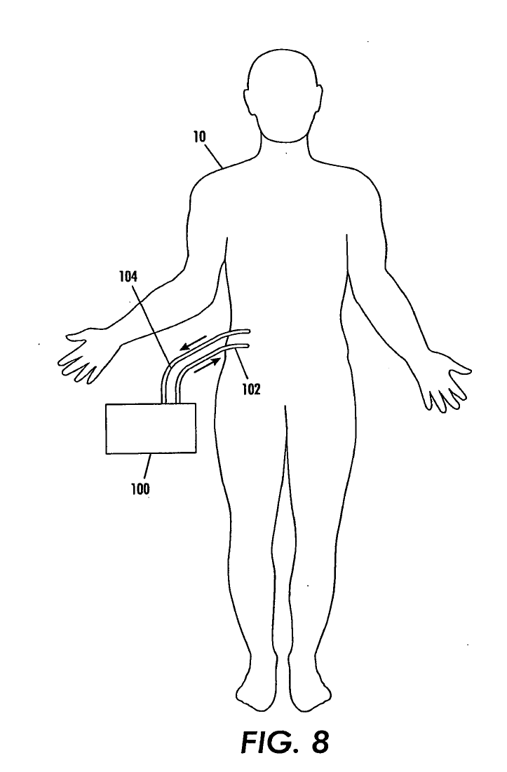

In the embodiment depicted in FIG. 8, bodily fluid is withdrawn from body 10 via line 104, treated with energy in device 100, and returned to the body via line 102. In this embodiment, a portion of the bodily fluid may be segregated in device 100 and treated separately from the other bodily fluid. Thus, e.g., the device 100 may comprise a flow cytometer which identifies cancerous cells, segregates them, treats them with high heat and/or radiation, and returns some or all of the cells so treated to the body.

In one embodiment, in any or all of the processes of this invention, the electromagnetic energy is delivered directly into one or more bodily fluids, such as, e.g., the blood, the lymph, the urine, cerebrospinal fluid, endolymph, aqueous humor, etc. Reference may be had, e.g., to FIG. 8, in which a bodily fluid is treated in reservoir 100 after being removed from a body 10 and then returned to such body 10.

FIG. 9 is a flow diagram of one process. In step 102 of the process, the emitter controller 16 (not shown) checks the blood pressure of the biological organism using, e.g., sensors 46 (see FIG. 4). If the blood pressure of the organism is lower than a specified level, in step 104 the process is aborted. If the blood pressure of the organism is higher than such specified level, then in step 106 the controller (not shown) optionally checks other body parameters (such as, e.g., body temperature, pulse rate, etc.) to determine whether it is safe to apply to specified therapy.

After verifying that the therapy regimen is safe, in step 108, millimeter wave frequency is applied for a specified duration such as, e.g., 15 minutes. Thereafter, the blood pressure of the biological organism is again checked in step 102′. In one aspect of this embodiment, if the blood pressure of the organism is still too high after the initial treatment, additional incremental treatments 110 preferably are continued up to a threshold decision point 112. In the embodiment depicted, additional chemical therapy is administered in step 114, and monitored in step 102″. If this additional drug therapy is not effective, the patient is alerted in step 118.

It will be apparent to those skilled in the art that the process depicted in FIG. 9 can have constructive application for a variety of other medical conditions besides the amelioration of high blood pressure. For example, another embodiment is in the regulation of carbohydrate metabolism in the diabetic patient. Here the sensor in step 102 monitors the concentration of glucose in the blood and millimeter or other frequencies are emitted in step 108 to effect a stimulation of glucose absorption in the tissues in the body. Again, if the blood glucose concentration in the organism is still too high after the initial treatment, additional incremental treatments 110 preferably are continued up to a threshold decision point 112. In the embodiment depicted, additional electromagnetic energy is administered in step 108 or additional chemical therapy is administered in step 114, and monitored in step 102″. If this additional electromagnetic or drug therapy is not effective, the patient is alerted in step 118. The entire configuration, or suitable variations of it, constitute what has been termed an “artificial pancreas.”

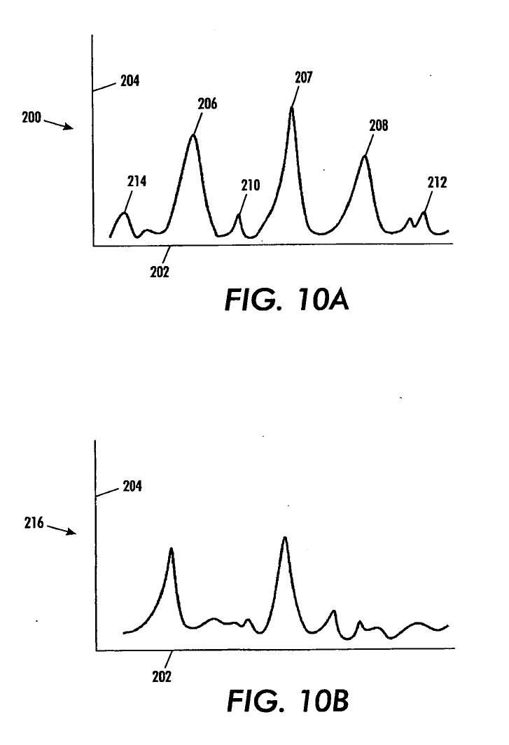

FIG. 10A is a graph of a spectrum 200 of one energy pattern delivered from the emitter 16 to a patient 10 at “time zero.” In the graph of this FIG. 10A, frequency is plotted on the horizontal axis 202, and amplitude is plotted on the vertical axis 204.

Referring to the graph depicted in FIG. 10A, it will be seen that the spectrum 200 is comprised of major peaks 206, 207 and 208 and minor peaks 210, 212, and 214. In general, the spectrum of the energy emitted by emitter 16, in this embodiment, will contain at least two major peaks and two minor peaks.

The spectrum 216 depicted in the graph of FIG. 10B is illustrative of the pattern emitted by the same emitter 16 at some time, t1, after “time zero.” As will be apparent, in this embodiment, the spectrum 216 differs from the spectra 200.

When a drug is administered to patient, its spectrum changes as it is dissolved within the patient's system and/or is metabolized within the patient. As the drug undergoes physical and/or chemical changes, its spectrum changes. In one embodiment of this invention, the energy pattern delivered by the emitter 16 is substantially comparable to the energy pattern delivered by a drug as it undergoes physical and/or chemical changes within the patient's body.

One may, by conventional techniques, measure the spectrum of one or more drugs as they interact with and within a patient's body. Thereafter, one may program this spectrum into an emitter comprised of programmable computer such that the emitter will deliver the same energy pattern to a biological organism as the drug did, over time. Thus, e.g., one may use the emitter 16 and the controller 44, as depicted in FIG. 4.

It will be apparent to those skilled in the art that the process just described may not be ideal, as alterations in the structure of drug molecules, and resulting alterations in the emission spectrum of the molecules, may be detrimental to the organism, leading to undesired side effects. Hence in another embodiment the computer is programmed such that the emitter will continue to deliver the same energy pattern to a biological organism as the drug did when the drug was first administered to the patient.

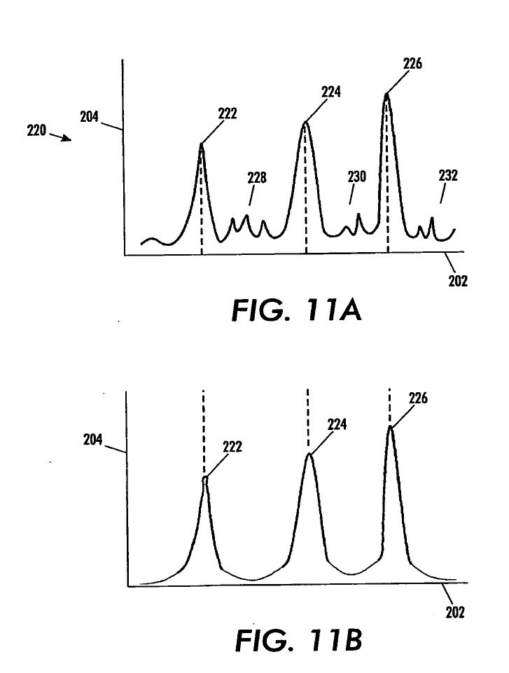

FIG. 11A illustrates a spectrum 220. In the particular embodiment depicted in FIG. 11A, and for a particular condition, it might be determined that only major peaks 222, 224, and 226 produce advantageous results but that the minor peaks in the “troughs” of the spectra, regions 228, 230, 232, have deleterious effects. In this case, as is illustrated in FIG. 11B, the controller 44 will cause emitter 16 to emit only the major peaks 222, 224, and 226. Alternatively, it may be determined that one or more of the major peaks is the cause of deleterious effects, in which case these major peaks are removed from the emitted spectrum.

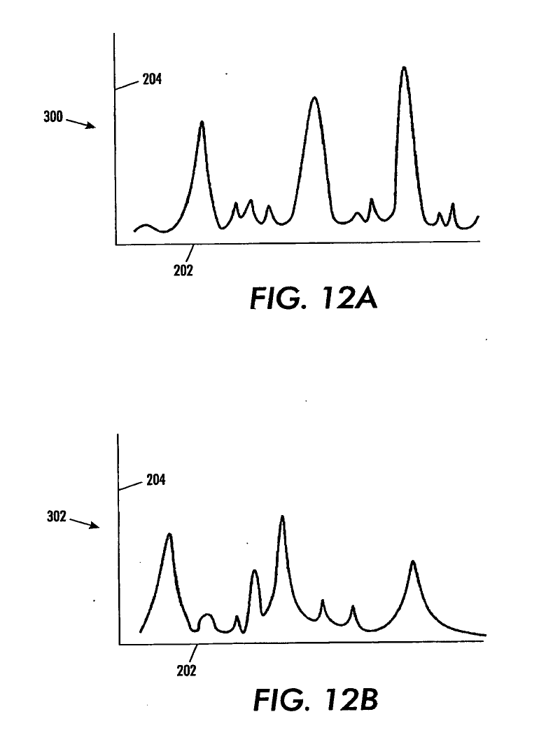

The process of this invention is not limited to the use of only one emitter 16 or only one implantable drug dispenser 240. As will be apparent to those skilled in the art, the use of a multiplicity of emitters 16 allows one to produce a large variety of different waveforms and spectra patterns that can interact with a multiplicity of injected drugs. FIGS. 12A and 12B illustrate the spectral patterns 300 and 302 which may be produced at one particular point in time by emitters 16 and 64 (see FIG. 5).

In one embodiment, there is provided an apparatus for treating a biological organism, comprising an externally worn and removable appliance comprised of means for inducing an electromagnetic and/or vibrational and/or light and/or other energy pattern of a biological process and/or a suitable drug or drugs through the skin of a organism which, preferably, is living. In this embodiment, the energy pattern corresponds to at least a portion of the electromagnetic pattern, or a modification thereof, of a biological process within the organism.

In many cases, it may be desirable to introduce more than one electromagnetic pattern to the patient. Thus, in one embodiment, depicted in FIGS. 7 and 12, there is provided a process comprising the steps of determining a first electromagnetic pattern of a biological process within a living organism, determining a second electromagnetic pattern of a biological process within a living organism, introducing said first electromagnetic pattern into said living organism, and introducing said second electromagnetic pattern into said living organism. As will be apparent, more than two such electromagnetic patterns may be administered, and they may be administered in combination with one or more drugs.

In one embodiment, in any or all of the processes of this invention, the electromagnetic energy and/or other energy is delivered directly into cartilage. In another embodiment of the invention, the electromagnetic and/or other energy is delivered directly into bone. In yet another embodiment of the invention, the electromagnetic and/or other energy is delivered directly into brain cells. In yet another embodiment of this invention, the electromagnetic and/or other energy is delivered to fascia and/or cerebrospinal fluid and/or other fluids. In yet another embodiment of this invention, the electromagnetic and/or other energy is delivered to acupuncture and/or other biologically active points within and/or on the body.

In any or all of the aforementioned embodiments, one may substitute for part or all of the electromagnetic energy other energy forms, such as vibratory energy.

After a suitable number of correlations have been made with the devices of this invention, one may deliver one or more energy patterns, and/or drugs, adapted to provide anti-allergy signals, anti-aids recognition signals, signals that reduce the side effects of drugs, signals that mimic the signals of homeopathic remedies, signals that mimic the patterns of heat drugs (such as beta blockers), nitrolycerine, anti-tumor drugs, antibiotics, antiviral agents, stress reducing agents, pain killers, and the like. As will be apparent, this list is merely illustrative.

In one embodiment, a desired electromagnetic spectrum and/or modulated light or sound (including, e.g., ultraviolet light or ultrasound or infrared radiation, e.g.) is injected directly into a patient's blood stream on demand and/or at regular intervals and/or continuously.

In one embodiment, the spectral pattern which exists when the AIDS virus attaches to a lymphocyte is determined, and a pattern designed to interfere with this first spectral pattern is emitted. Thus, e.g., one may emit coherent photon signals that mediate the behavior of the AIDS viron and its attraction to and identification of and docking on the human lymphocyte. In one aspect of this embodiment, either the viron itself and/or a component of the viron is caused to resonate at its natural coherent resonant frequency. Two key elements of such viron are two surface proteins, glycoprotein GP41 and glycoprotein GP 120; they constitute a dielectric antenna. By the application of suitable electromagnetic energy to such “antenna,” the AIDS viron can be affected.

In one embodiment, the emitter 16 is comprised of means for transmitting a desired electromagnetic pattern to a pacemaker. Thus, e.g., one may transmit suitable analog, digital, or scalar versions of such signals to a cardiac assist device. In one aspect of this embodiment, the cardiac assist device is adapted to store the spectrum transmitted to it by the emitter 16 and, when appropriate, to retransmit part or all of such spectrum.

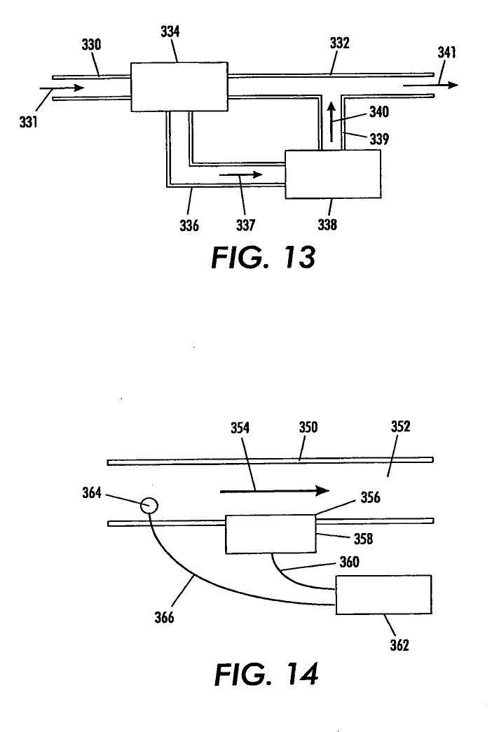

In another embodiment, see FIG. 13, an emitter is built into a shunt. Referring to FIG. 13, a blood artery or vein is divided into two parts 330, 332 and a filter/separator 334 is inserted between them. Arrows 331, 337, 339, 341 show the direction of fluid flow. The filter/separator 334 diverts a portion for the plasma into line 336. That portion of the blood fluid which is not diverted to 336 is returned to the artery or vein 332. The divert plasma enters an emitter chamber 338 where the energy pattern is applied to the plasma. Said energy pattern may be millimeter wave, acoustic energy, light, etc. as describe throughout this disclosure. The treated plasma returns to the artery or vain through line 340. By way of illustration, but not limitation, 334, 336, 338 may be components of an artificial heart implant into the body.

In another embodiment, fluid is treated externally and independent of a body as it flows through tubing. In FIG. 14, tubing 350 carrying a fluid 353 flowing in the direction 354 has an emitter 358 implanted through the tubing wall so that the emitter tip 356 is in contact with the fluid 352. By way of illustration, but not limitation, said tubing 350 may be the fuel line of a vehicle, the water supply line to a faucet, the outlet of a water dispenser, an intravenous (IV) line, an implanted stint, etc. The emitter 358 is connected to a controller 362 by communications means 360 which may be, e.g., a wire, fiber optics cable, RF or other means. The controller 362 controls the type of energy, pattern of energy, application timing, duration, magnitude and/or other adjustable parameters. Additionally, a optional sensor 364 may be inserted into the tubing. Said sensor 364 may measure, e.g., the flow rate of the fluid 352 and/or the temperature of the fluid 352, the pH level of the fluid 352 and/or other measurable properties. Said sensor is connected to controller 362 by communication means 366 which may be, e.g. a wire, fiber optic cable, RF or other means.

In another embodiment (not shown), the emitter tip 356 of FIG. 14 is attached externally to the tubing wall 350. In this embodiment, the emitter tip 356 does not come into direct contact with the fluid 352.

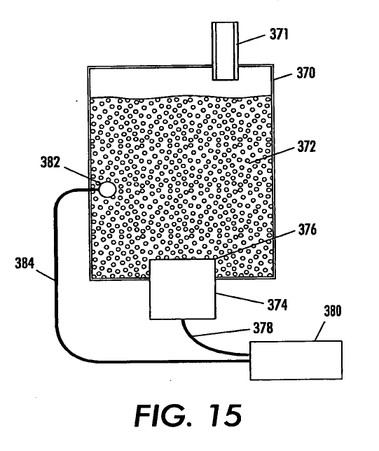

In another embodiment, see FIG. 15, the fluid 372 to receive the energy pattern treatment is preferably contained in a vessel 370 which has means 371 for removing and/or replenishing said fluid 372. By way of illustration and not limitation, said vessel 370 may be e.g., a hot water heater, a thermos or canteen, a coffee maker, an IV bag, a gasoline tank, etc. In one embodiment, emitter 374 has its emitting tip 376 in contact with fluid 372. In another embodiment (not shown) the emitter tip 376 is external to the vessel 370. In FIG. 15, the emitter 374 is connected to controller 380 via communication means 378 which may be, e.g., a wire, fiber optics cable, RF or other means. The controller 380 controls the type of energy, pattern of energy, application timing, duration, magnitude and/or other adjustable parameters. Additionally, a optional sensor 382 may be inserted into the vessel 370. Said sensor 382 may measure, e.g., the temperature of the fluid 372, the pH level of the fluid 372 and/or other measurable properties. Said sensor 382 is connected to controller 380 by communication means 384 which may be, e.g. a wire, fiber optic cable, RF or other means.

In yet another embodiment of the invention, a process for the treatment of disease, such as cancer is provided. Although the process is applicable to many different diseases, it will be described by reference to cancer for simplicity of description.

The group of diseases commonly referred to as cancer in fact includes a highly diverse set of cell types that have, through a process of mutation, begun a process of unregulated proliferation. Since the accumulation of these mutations is a random process, the combination of mutations that ultimately result in a cancerous disease state varies widely. This complicates the process of disease treatment, as each protocol must be tailor-made to suit each different patient.

Physicians have long sought a treatment for cell proliferation diseases (such as cancer) that could be generalized for the treatment of all of these related maladies, avoiding this process of “tailoring making” a protocol that may involve invasive diagnostic techniques that can be uncomfortable for the patient and rely on conventional pathological analysis which is expensive, time-consuming and often is based on techniques that have variable accuracy.

One unique property of cancer cells is their ability, once in their fully transformed state, to become motile. This property is known to those of skill in the art as invasive and metastasis.

In one embodiment, describe more fully elsewhere in this specification, there is disclosed a process and device to influence the cell motility and cell division cycle of cancer and other diseased cells in order to slow or stop their proliferation and thereby slow the progression of the disease or affect a cure. As will be apparent to those skilled in the art, disease processes involving the control of cell proliferation include, but are not limited to restenosis of vascular and arteriole tissue following angioplasty or the introduction of a vascular stent, the development of excessive or unwanted scar tissue, thickening of ventricular walls in hypertrophic cardiomyopathy, angiogenesis of tumor masses, psoriasis, and other related disorders.

In one embodiment of this invention, also described elsewhere in this specification, infrared light is used to promote the migration of cells or cell appendages to a particular region. This migration can include, but is not limited to, angiogenesis, nerve axons, and myocytes.

In one embodiment of this invention, pulsed infrared light is used to turn away invasive or migrating cells from an irradiated area. In another embodiment, light in the visual and ultraviolet spectrum is used. Each of these embodiments is described elsewhere in this specification.

In another embodiment, non-cancer and/or cancer cells are cultured by methods routine to those skilled in the art, and exposed to light in the range of 660-1000 nanometers with varied pulse rates and wavelengths that were discovered to be capable of altering the progression of the cell cycle. This alteration could be a cessation or a signal to begin cell division. In another embodiment, light in the visual and ultraviolet spectrum is used.

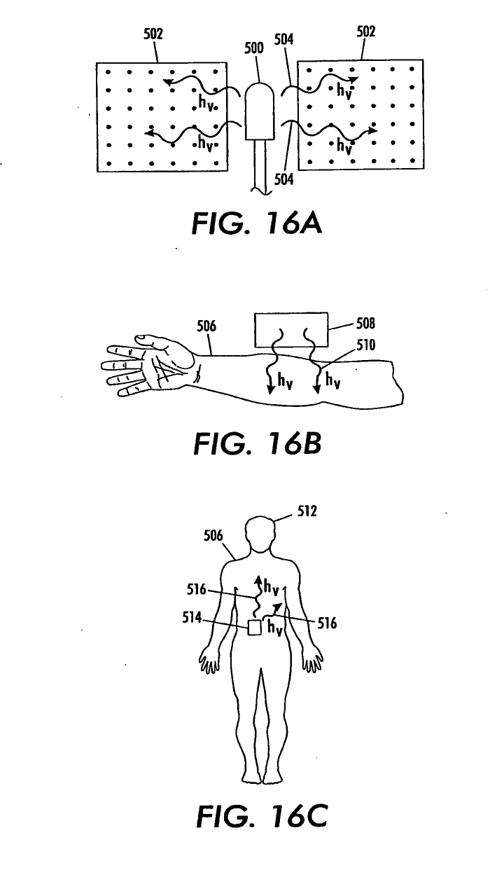

FIG. 16A illustrates an intravascular probe 500 that is capable of delivering light, 504, of various wavelengths to tissues 502. FIG. 16B represents an external device 508 that delivers light energy, 510, to a patient 506. The use of this device is not limited to any appendage or target organ in particular. FIG. 16C represents an implantable version of a light emitting device 514 that can deliver light energy 516 to the patient 512.

In another section of this specification, various types and regimens of energy are delivered to a biological organism. It will be understood that any one or more of these energy protocols may be used with one or more of the devices of FIGS. 16A, 16B and/or 16C.

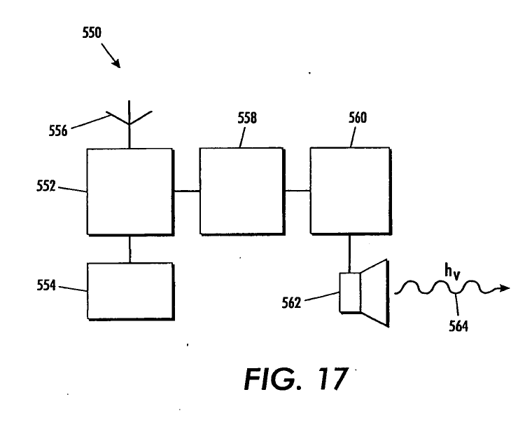

FIG. 17 is a diagram of another device 550 that may be used with one or more of such energy protocols. Referring to FIG. 17, and to the embodiment depicted therein, it will be seen that device 550 may be used for the delivery of light energy 564 to a patient or cell system in vivo or in vitro. In one embodiment, the device 550 is comprised of a main control unit 552 powered by an electrical power supply 554. An antenna 556, or antennae 556 allows for the telemetric reception of control signals from the exterior of the body, in the case of an implantable device, or other remote control in the case of external devices, and for the transmittance of information from the device to the user or caregiver using standard means of such radio transmission, known to those familiar with the art.

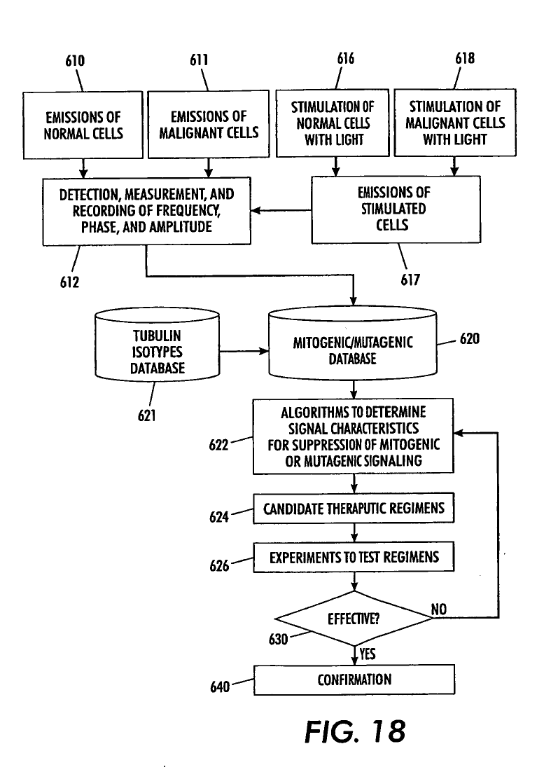

In one embodiment, the control unit 552 uses information derived from the process described in FIG. 18 to operate solenoid 558. Solenoid 558 is preferably responsible for the temporal modulation of the signal, the pulse, as referred to above, produced by the transducer 560. Transducer 560 produces a frequency of light that is focused or dispersed (depending on the required regimen) by lens/reflector assembly 562 to produce the emission 564, the biologically active signal that is ultimately delivered to the organism or biological system.

In one embodiment the properties of the light emitted by one or more of the devices of FIGS. 16A, and/or 16B, and/or 16C, and/or 17 are described below.

In one embodiment, the wavelength is between about 601 and about 1200 nanometers. In another embodiment the wavelength is from about 390 to about 600 nanometers. In yet another embodiment, the wavelength is from about 200 to about 389 nanometers. In one aspect of these embodiments, regardless of the wavelength used, the light energy is pulsed into a biological sample (such as a tissue sample) at a 0.5 hertz pulse rate. In another aspect of this embodiment, the pulse rate is from about 0.55 hertz to about 0.7 hertz. In another aspect of this embodiment, the pulse rate is less than about 0.4 hertz and preferably is from about 0.1 to about 0.4 hertz. In another embodiment, when using one or more of the aforementioned wavelengths of light, the light is continuous.

FIG. 18 is a flow diagram of a process for treating cells of biological organisms. Referring to FIG. 18, and to the embodiment depicted therein, electromagnetic emissions from normal and malignant cells (see elements 610 and 611, respectively), are detected, measured, compared, and stored in a database (see step 612 and element 620). The differences and frequency and phase-coherence relationships between the normal and malignant cells, both for individual patients and tubulin isotypes, are determined by analysis of this data 620. Based on this analysis, a plurality of different therapeutic regimens are then developed and tested (see steps 622, 624, 626, and 630) to determine the optimal regimens for particular patients and particular tubulin isotypes.

Referring again to FIG. 18, and in step 612 thereof, electromagnetic emissions, preferably in the range of 1 GHz to 1 teraHertz, from both normal cells 610 and malignant cells 611, are detected, measured, and recorded in step 612 to determine relevant measures of the biological signals such as, but not limited to, spectral density and phase coherence.

Spectral density and phase coherence of the emissions from normal cells 610 and malignant cells 611 are computed in step 612 and stored in database 620. Such detection and measurement can be accomplished by well-known electronic devices; e.g., spectrum analyzers and oscilloscopes.

Optionally, and in one embodiment, Fast Fourier transform algorithms may be used with wideband signals to determine power spectral density of biological data by converting a signal in the time domain into data in the frequency domain, using either digital signal processors or the equivalent algorithms in software.

As is known to those skilled in the art, such measurements of spectral density and phase coherence may be performed in a Faraday cage to attenuate artifacts from environmental sources. In addition, artifacts from electrical signals in the extreme low frequency (ELF) range, such as 60 Hz power line signals, may be attenuated by using battery-powered equipment, mu-metal shielding, common-mode-rejection circuits, and other methods.

Optionally, and in one embodiment, electromagnetic signals in the 100 to 1200 nanometer wavelength range are detected, measured, and recorded for normal cells 610 and malignant cells 611. One may detect, measure, and record spectral density and phase coherence for such signals by well-known devices; e.g., spectrophotometers, photomultipliers, and the like, or combinations thereof.

Referring again to FIG. 18, and in steps 616 and 618 thereof, normal cells and malignant cells are stimulated by brief exposure to light in multiple wavelength increments throughout the 100 to 1200 nanometer range. Stimulated emissions from the normal and malignant cells are then detected and measured in step 617 to determine their degree of coherence, as described by Popp, F. A. and Li, K. H. in “Hyperbolic relaxation as a sufficient condition of a fully coherent ergodic field” in Recent Advances in Biophoton Research (F. A. Popp, K. H. Li and Q. Gu, eds.), pp. 47-58, World Scientific, Singapore. These data are stored in database 620.

The aforementioned steps (shown in 610, 611, 612, 616, 617, 618) are also repeated to determine the signature of electromagnetic radiation for normal and malignant cells for each isotope in the database of tubulin isotypes in step 621.

The signature (unique patterns) of the spectral density and phase coherence of the electromagnetic radiation for each group of normal and malignant cells is then determined by computational algorithms (which can be executed in either hardware or software) known to those skilled in the art, based on an analysis of the normal and malignant electromagnetic radiation and other data in the database.

Referring again to FIG. 18, and in step 622 thereof, based on this signature, as well as information gathered from the patient using conventional means that include, but are not limited to, a characterization of the predominant tubulin isotypes 621 present in the tumor cells of the patient in question, one or more algorithms 622 (which can be executed in either hardware or software) generate candidate therapeutic frequency and phase regimens 624 that are most effective in suppressing mitogenic or mutagenic signaling for malignant cells, using electromagnetic emissions with a plurality of pulse modulations, amplitude modulations, frequency modulations, phase modulations, pulse trains, and combinations of one or more these techniques.

Referring again to FIG. 18, candidate therapeutic frequency and phase regimens are determined that are most effective as electronic countermeasures (ECM). Reference may be had to ATIS Telecom Glossary 2000, which defines “electronic countermeasures” as “That division of electronic warfare involving actions taken to prevent or reduce an enemy's effective use of the electromagnetic spectrum.”

In one embodiment, after a measurement is made of the emission(s) from the normal cells and/or the cancer cells, phase-cancellation signals are sent out to selectively confuse or incapacitate the cancer cells. Thus, e.g., one may use real-time phase cancellation, as known to those skilled in the art. Phase cancellation is achieved by transmitting an inverse (180 degrees out of phase) signal at the same frequency as a detected mitogenic or mutagenic signal. As a result, the mitogenic or mutagenic signal may be attenuated or blocked.

For example, mitogenic or mutagenic signals may be blocked using electronic countermeasures techniques such as, for example, electromagnetic radiation (at microwave or optical frequencies) that is modulated with high levels of noise (“jamming”) at target-sensitive frequencies or ranges of frequencies, or at specific power levels, or by specific pulse trains, or at specific phase regimens, or by synchronizing with mitogenic signals, or by using phase cancellation with mitogenic signals, or by using pulse trains that confuse mitogenic or mutagenic signals, or by using pulse trains that confuse by signalling completion of an event such as mitosis, or by any combination of these tactics, or by using a plurality of other electronic countermeasures techniques that are well known to those skilled in the art.

The candidate therapeutic regimens 624 are then executed in designed experiments 626 seeking to determine the optimal methods of entraining orderly cell division and appropriate coherence in electromagnetic energy emitted by cells. The effects of the candidate regimens are then measured, using standard techniques for the assessment of tumor mass growth, regression and remission known to those of skill in the art. In step 630 of such designed experimentation, a specific regimen that produces the desired therapeutic result for the patient, if successful, is determined and confirmed in final step 640.

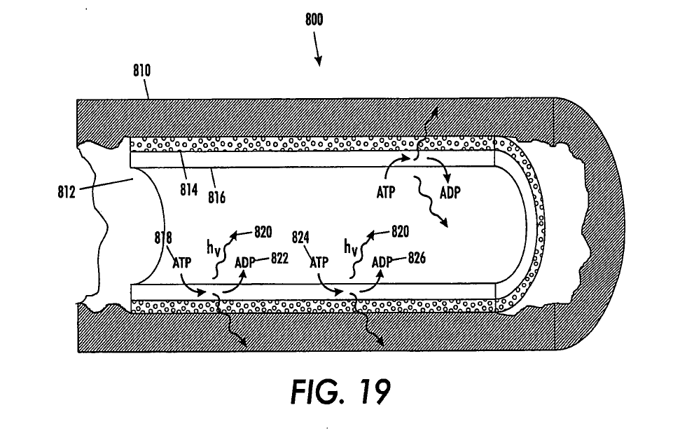

FIG. 19 is a schematic illustration of a coated stent 800 coated with a light emitting coating 810 for preventing restenosis.

Referring to FIG. 19, and in the preferred embodiment depicted therein, there is shown a cross sectional diagram of a blood vessel 810 that is being held open by a vascular stent 812. The stent is preferably constructed of a metal or plastic material 814 with a coating 816 disposed thereon. Coating 816 preferably contains a chemiluminescent material that converts sources of chemical energy, such as, e.g., ATP 818, available in the circulation, into a reduced form, ADP 822 and, in the process, produces light, 820. Chemiluminescent materials are well known to those skilled in the art.

Referring again to FIG. 19, the chemical energy source, generically labeled 824, is not exclusively ATP but could be NADPH, glucose, pyruvate, or other available chemical energy sources. The reduced or other product of this reaction is generically referred to as P in FIG. 19 and is identified with numeral 826. This chemical reaction results in the release of light 820. In one preferred embodiment, the wavelength of the produced light is in the near-infrared part of the spectrum, i.e., about 800 to about 1200 nanometers, and interacts with the cells of the smooth muscle layer 810 of the vessel wall to inhibit cell migration into the interior of the stent, a process called restenosis. In another preferred embodiment, the wavelength of light released during this reaction is in the visible part of the spectrum, i.e., about 400 to about 800 nanometers.

In one preferred embodiment, described elsewhere in this specification, the wavelength of the light used is a wavelength determined a process described elsewhere to be toxic to cancer cells.

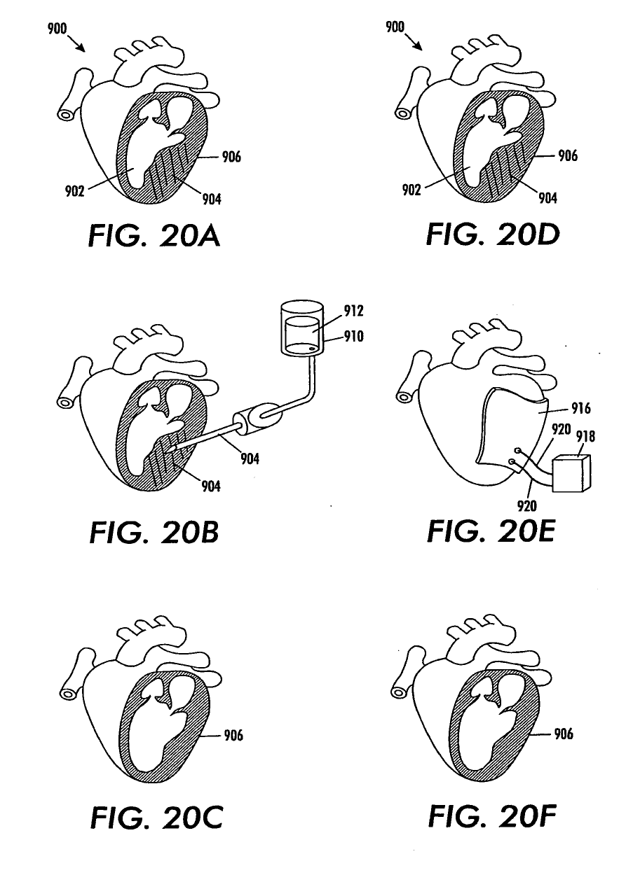

FIGS. 20A-F is a schematic diagram of an process for treatment of congestive heart failure.

Referring to FIGS. 20A-F There is shown a cross-sectional diagram of a human heart 900 with the left ventricle 902 partially occluded by hypertrophic cardiomyopathy 904 of the ventricle wall 906 of the heart. Hypertrophic cardiomyopathy, as is well known to those skilled in art, is a disease caused by the disorganized and inappropriate proliferation of cells of the wall 906 of the left ventricle 902 and leads to a debilitating and potentially fatal condition commonly known as congestive heart failure. FIG. 20A shows the diseased heart 900 before intervention. FIG. 20B shows the hypertrophic region 904 of the heart being penetrated with a syringe 908 and injected with an inoculant 912 contained in a vessel 910. Elements 908 and 910 are not necessarily drawn to scale. The inoculant 912 contains a quantity of genetically engineered, reproduction incompetent, viruses capable of infecting the hypertrophic cells. This virus preferably contains a gene capable of expressing a protein in a eukaryotic system that is chemiluminescent; that is, it uses energy sources in the cell to produce light. In one preferred embodiment, the wavelength of the produced light is in the near-infrared part of the spectrum, i.e., about 800 to about 1200 nanometers, and interacts with the cells of the ventricular wall 906 and the aberrant hypertrophic cells 904, to inhibit cell migration and cell division into the interior of the left ventricle 902. In another preferred embodiment, the wavelength of light released during this reaction is in the visible light part of the spectrum, i.e., about 400 to about 800 nanometers. This reduction of cell proliferation and migration results in the restoration of the ventricular wall 906 to its normal, non-diseased size, restoring function and successfully treating the congestive disease state.

In another preferred embodiment of this invention, and with reference to FIG. 20D, a hypertrophic heart is fixed with a patch or cover, 916 (see FIG. 20E). This patch is connected to control and power unit 918 by wires 920, or, in another preferred embodiment, is self powered by chemical energy sources in the thoracic or by the kinetic motion of the heart. This patch is capable of the emission of light energy (as described in previously in this disclosure) in order to slow or prevent the enlargement of the hypertrophic growth, 904, of the ventricle wall, 906. In an additional preferred embodiment, the energy emitted by the patch includes sources of energy other than light energy. In another preferred embodiment, the patch gathers information from the heart and is able to telemetrically communicate this information to a clinician caring for the patient. This monitoring of the cardiac function includes, but is not limited to, the collection of sound information. As will be apparent to those skilled in the art, hypertrophic disease causes a change in, among other measurable conditions that will be apparent to those skilled in the art, the sound of the blood moving through the heart chambers and the valves between them. By constantly monitoring the sound, or other conditions, of the heart, heart failure, murmurs or infarction or other conditions may be detectable in the early stages, before a potentially fatal cardiac crisis is underway. Control unit 918 could then inform the clinician, patient or emergency medical team that acute medical intervention is required. This telemetric communication with heath care providers includes, but is not limited to wireless devices and technologies such as “Bluetooth,” cell phone or satellite locating and warning systems known to those of skill in the art.

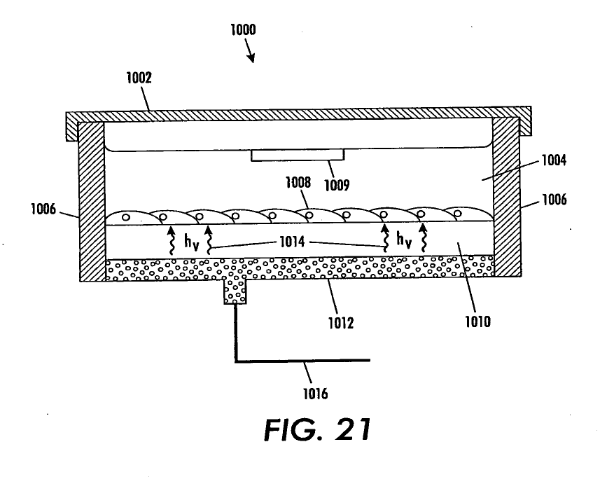

FIG. 21 is a schematic illustration of a device 1000 for interrogating cellular components. The cellular components to be interrogated include, e.g., centrioles, microtubules, actin and actin-containing structures, tubulin and tubulin-containing structures, proteins of membrane or the cytoplasm or the mitochondria, and the like.

Referring again to FIG. 21, and to the preferred embodiment depicted therein, the cellular components are disposed in a solution containing gelatin, agarose, or other solid or semi-solid media capable of supporting a eukaryotic cell monolayer, such as monolayer 1008. The eukaryotic cell monolayer may be, e.g., a monolayer transformed or non-transformed immobilized cell lines, or cells of primary culture. In one embodiment, eukaryotic cell monolayer is 3T3 cells, COS-1 cells, C6 cells, and the like.

In the embodiment depicted, the monolayer 1008 is disposed on the media 1010. A light emitting device 1012 is preferably disposed below the monolayer 1008, and it is adapted to emit one or more of the radiations described elsewhere in this specification.

In the preferred embodiment depicted, and referring again to FIG. 21, the light source 1012 is connected via line 1016 to a controller (not shown) that is adapted to control the emission produced by light source 1012 in a pattern that is either predetermined and/or is determined by one or more measured parameters.

Lid 1002 is configured so as to ensure an optically sterile environment; it is preferably opaque, neither allowing light to enter or leave. As used herein, the term light with a wavelength of from about 200 to about 1200 nanometers.

In the preferred embodiment depicted, the device 1000 is preferably shielded from radio frequency radiation by Mu metal shields 1006.

Referring again to FIG. 21, as light energy 1014 is emitted from light source 1012, it travels through the media 1010 and thereafter contacts the cellular components (not shown) disposed in media 1010. Without wishing to be bound to any particular theory, applicants believe that the cellular components disposed in media 1010, upon being contacted with light energy 1014, emit harmonics thereof. Thus, e.g., it the light source radiates energy 1014 with a wavelength of 800 nanometers, a second harmonic (400 nanometers) and a third harmonic (200 nanometers, and/or other harmonics) are emitted.

In one preferred embodiment, it is preferred to generate harmonics with a wavelength of from 200 to 400 nanometers. In this embodiment, one thus would utilize a light source 1012 that produced light with a wavelength of 400 to 800 nanometers.

After light energy 1014 has been emitted from the light source 1012, one can observe the effect of such light energy 1014 (and/or of the harmonics it creates) upon the cell monolayer 1008. One can remove the lid 1002 and observe whether the cells have proliferated, and/or been killed, and/or moved. In one embodiment, camera 1009 continually monitors the effects of the radiation 1014 upon the cell monolayer and transfers this information by a telemetric link (not shown) to the controller (not shown).

As will be apparent, the device 1000 allows one to determine the effects, if any, upon cellular health of various light regimens. Some of these are discussed elsewhere in this specification.

In one preferred embodiment, the light regimen in question preferentially kills cancer cell and/or preferentially stops the cell division of cancer and/or preferably stops the motility of cancer cells.

In one embodiment, the cell monolayer 1008 is a cell monolayer derived from cancer cells taken from a patient. As will be apparent, one can determine, for these particular cells in question, which light energy regimen is most efficacious in treating such cancer cells. Thereafter, one can implant a device, such as the device depicted in FIG. 16C, in the patient and program the device to direct the appropriate light therapy to the tumor in question. As will be apparent, cells contained in a hypertrophic left ventricle (see FIG. 20) may also be interrogated with this device 1000 so as to find a signal that is non-proliferative for hypertrophic versus normal myocytes. These frequencies can then be used in the device of FIG. 20E.

While the invention has been described with reference to certain embodiments, it will be understood by those skilled in the art that various changes may be made and equivalents may be substituted for elements thereof to adapt to particular situations without departing from the scope of the disclosure. Therefore, it is intended that the claims not be limited to the particular embodiments disclosed, but that the claims will include all embodiments falling within the scope and spirit of the appended claims.

What is claimed is:

1. A process for treating a biological organism, said process comprising the steps of:
measuring a first electromagnetic energy signal characteristic of a biological sample while experiencing the effect of a therapeutic agent;
measuring a second electromagnetic energy signal characteristic of the biological sample while not experiencing the effect of the therapeutic agent, wherein the first and second electromagnetic energy signals are different;
comparing the first and second electromagnetic energy signals and, based on this comparison, determining a third electromagnetic energy signal that, when superimposed on the second electromagnetic energy signal, results in a fourth electromagnetic energy signal that is substantially identical to the first electromagnetic energy signal; and
delivering the third electromagnetic energy signal to a biological organism which corresponds to the biological sample while the biological organism is not experiencing the effect of the therapeutic agent.

2. The method as recited in claim 1, wherein the step of measuring the first electromagnetic energy signal includes the step of subjecting the biological sample to an electromagnetic excitation field, wherein the first electromagnetic energy signal is produced in response to the electromagnetic excitation field.

3. The method as recited in claim 2, wherein the step of measuring the second electromagnetic energy signal includes the step of subjecting the biological sample to the electromagnetic excitation field, wherein the second electromagnetic energy signal is produced in response to the electromagnetic excitation field.

4. The method as recited in claim 1, wherein the biological sample is a heart and the steps of measuring the first and second electromagnetic energy signal are conducted as part of a Langendorff experiment.

5. The method as recited in claim 3, wherein the therapeutic agent is an anti-inflammatory drug.

6. The method as recited in claim 3, wherein the therapeutic agent is nabumetone.

7. A process for treating a human being, said process comprising the steps of:
measuring a first electromagnetic energy signal characteristic of a human tissue sample while experiencing the effect of a therapeutic agent;
measuring a second electromagnetic energy signal characteristic of the human tissue sample while not experiencing the effect of the therapeutic agent, wherein the first and second electromagnetic energy signals are different;
comparing the first and second electromagnetic energy signals and, based on this comparison, determining a third electromagnetic energy signal that, when superimposed on the second electromagnetic energy signal, results in a fourth electromagnetic energy signal that is substantially identical to the first electromagnetic energy signal; and
delivering the third electromagnetic energy signal to a human being while the human being is not experiencing the effect of the therapeutic agent.

8. The method as recited in claim 7, wherein the step of measuring the first electromagnetic energy signal includes the step of subjecting the human tissue sample to an electromagnetic excitation field, wherein the first electromagnetic energy signal is produced in response to the electromagnetic excitation field.

9. The method as recited in claim 8, wherein the step of measuring the second electromagnetic energy signal includes the step of subjecting the human tissue sample to the electromagnetic excitation field, wherein the second electromagnetic energy signal is produced in response to the electromagnetic excitation field.

10. The method as recited in claim 9, wherein the electromagnetic excitation field has a frequency range lower than 100 kHz.

11. The method as recited in claim 9, wherein the electromagnetic excitation field has a frequency range above 1 Hz and less than 20 kHz.

12. A process for treating a human being, said process comprising the steps of:
measuring a first electromagnetic energy signal characteristic of a human tissue sample while experiencing the effect of ibuprofen;
measuring a second electromagnetic energy signal characteristic of the human tissue sample while not experiencing the effect of the ibuprofen, wherein the first and second electromagnetic energy signals are different;
comparing the first and second electromagnetic energy signals and, based on this comparison, determining a third electromagnetic energy signal that, when superimposed on the second electromagnetic energy signal, results in a fourth electromagnetic energy signal that is substantially identical to the first electromagnetic energy signal; and
delivering the third electromagnetic energy signal to a human being while the human being is not experiencing the effect of the ibuprofen.

13. The method as recited in claim 12, wherein the step of measuring the first electromagnetic energy signal includes the step of subjecting the human tissue sample to an electromagnetic excitation field, wherein the first electromagnetic energy signal is produced in response to the electromagnetic excitation field.

14. The method as recited in claim 13, wherein the step of measuring the second electromagnetic energy signal includes the step of subjecting the human tissue sample to the electromagnetic excitation field, wherein the second electromagnetic energy signal is produced in response to the electromagnetic excitation field.

15. The method as recited in claim 14, wherein the electromagnetic excitation field has a frequency range lower than 100 kHz.

16. The method as recited in claim 14, wherein the electromagnetic excitation field has a frequency range above 1 Hz and less than 20 kHz.
